(19) United States
(12) Patent Application Publication
(10) Pub. No.: US 2025/0345602 A1
(43) Pub. Date: Nov. 13, 2025

(54) DELIVERYBIPOLAR CURRENT DELIVERY APPARATUS AND METHOD VIA SKIN

(71) Applicant: SKINGRAB CO., LTD., Seoul (KR)

(72) Inventor: Sugun LEE, Paju-si (KR)

(73) Assignee: SKINGRAB CO., LTD., Seoul (KR)

(21) Appl. No.: 19/204,028

(22) Filed: May 9, 2025

(30) Foreign Application Priority Data
May 9, 2024 (KR) 10-2024-0061213

(51) Int. Cl. A61N 1/32; A61N 1/05; A61N 1/36

(52) U.S. Cl. CPC A61N 1/328; A61N 1/0502; A61N 1/36017; A61N 1/36034

(57) ABSTRACT

Disclosed are an apparatus and a method for delivery a bipolar current through skin contact. The bipolar current delivery apparatus includes an electrode unit including a plurality of first electrodes of a needle-shaped and a plurality of second electrodes of a needle-shaped which are inserted into or in contact with a skin, an energy supply unit that applies a first current of a bipolar type to the first electrodes and applies a second current of a bipolar type to the second electrodes, and a control unit that controls the energy supply unit such that a first frequency for the first current and a second frequency for the second current are set independently of each other. The first frequency and the second frequency are different from each other.

### CROSS-REFERENCE TO RELATED APPLICATIONS

This application claims the benefit of priority under 35 U.S.C. § 119 to Korean Patent Application No. 10-2024-0061213, filed on May 9, 2024, in the Korean Intellectual Property Office, the entire contents of which are hereby incorporated by reference.

### BACKGROUND

Embodiments of the present disclosure described herein relate to an apparatus and a method for supplying a bipolar current through skin contact, and more particularly, relate to an apparatus and a method for supplying a bipolar current that are capable of simultaneously supplying currents of various frequencies.

In general, skin care apparatuses for removing wrinkles, restoring skin elasticity, and removing sebum use a method (HIFU type) of delivering ultrasound to skin tissues, a method (RF type) of delivering high-frequency waves to skin tissues, and a method (Optical type) of irradiating laser light to skin tissues.

The apparatus that delivers high-frequency waves to skin tissues may repeatedly invade a deep portion (e.g., a dermis layer) of the skin (e.g., a face) with an RF needle electrode moving back and forth in a vertical direction, may remove damaged collagen, elastic fibers, or the like from the deep portion of the skin at a target point by using the heat generated by high-frequency waves, and may promote new formation.

Furthermore, the skin care apparatus improves skin pigmentation, acne scars, and wrinkles.

In other words, after a variety of energy is applied to a target area of the skin to intentionally cause wounds, the target area of the skin is regenerated by stimulating collagen in the dermis layer and inducing collagen regeneration.

However, the conventional skin care apparatuses have limited the range of skin depth in which electric energy is capable of being delivered. Accordingly, it was impossible to deliver electrical energy to the entire skin layer rather than just treating specific areas of the skin.

Therefore, the conventional skin care apparatuses may not deliver electric energy to the entire skin layer during a procedure, thereby somewhat reducing the treatment effect.

There is a prior art disclosed as Korean Registered Patent Publication No. 10-2556792.

### SUMMARY

Embodiments of the present disclosure provide an apparatus that may deliver electric energy to the entire skin layer during a procedure, thereby enhancing the therapeutic effect.

Embodiments of the present disclosure provide an apparatus that may simultaneously or alternately apply currents of various frequencies to a skin.

Problems to be solved by the present disclosure are not limited to the problems mentioned above, and other problems not mentioned will be clearly understood by those skilled in the art from the following description.

According to an embodiment, a bipolar current delivery apparatus includes an electrode unit including a plurality of first electrodes of a needle-shaped and a plurality of second electrodes of a needle-shaped which are inserted into or in contact with a skin, an energy supply unit that applies a first current of a bipolar type to the first electrodes and applies a second current of a bipolar type to the second electrodes, and a control unit that controls the energy supply unit such that a first frequency for the first current and a second frequency for the second current are set independently of each other. The first frequency and the second frequency are different from each other.

In an embodiment, the control unit may control the energy supply unit such that a current is simultaneously applied to the first electrodes and the second electrodes.

In an embodiment, the control unit may control the energy supply unit such that a current is alternately applied to the first electrodes and the second electrodes.

In an embodiment, the bipolar current delivery apparatus may further include a plurality of ground electrodes of a needle-shaped that are inserted into or in contact with a skin. The first electrodes and the second electrodes may be placed at edges of an area where the ground electrodes are placed.

In an embodiment, the first electrodes and the second electrodes may be placed alternately with each other.

In an embodiment, the bipolar current delivery apparatus may further include at least one switching electrode of a needle-shaped that is inserted into or in contact with a skin. The control unit may control the energy supply unit such that a current applied to the switching electrode is changed from one of the first current and the second current to the other.

In an embodiment, the switching electrode may be placed in a central portion of the area where the ground electrodes are placed.

According to an embodiment, a method performed by a bipolar current delivery apparatus including a plurality of first electrodes of a needle-shaped and a plurality of second electrodes of a needle-shaped which are inserted into or in contact with a skin includes setting a first frequency for a first current applied to the first electrodes, applying a first current of a bipolar type to the first electrodes, setting a second frequency for a second current applied to the second electrodes, and applying a second current of a bipolar type to the second electrodes. The first frequency and the second frequency are independently set to different frequencies.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a drawing showing a configuration of a bipolar current delivery apparatus, according to an embodiment of the present disclosure;

FIG. 2 is a drawing showing an electrode unit of FIG. 1 provided in cartridge housing coupled to a handpiece;

FIG. 3 is a drawing showing an example of a process in which a plurality of electrodes of a needle-shaped invade the inside of a skin by movement of the transfer unit of FIG. 1;

FIG. 4 is a drawing showing an embodiment of the placement of a plurality of electrodes included in the electrode unit of FIG. 1;

FIG. 5 is a drawing showing an example of a process of applying a first current and a second current of a bipolar type to a plurality of electrodes of the needle-shaped of FIG. 4; and

FIGS. 6 to 14 are diagrams showing various current and frequency patterns that are capable of being applied by a bipolar current delivery apparatus according to an embodiment of the present disclosure.

### DETAILED DESCRIPTION

Like reference numerals denote like elements throughout the drawings. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

In this specification, a control unit of a bipolar current delivery apparatus through skin contact according to an embodiment of the present disclosure includes all various devices capable of providing results to a user by performing arithmetic processing. For example, the control unit of a bipolar current delivery apparatus according to an embodiment of the present disclosure may include all of a computer, a server device, and a portable terminal, or may be in any one form.

Here, for example, the computer may include a notebook computer, a desktop computer, a laptop computer, a tablet PC, a slate PC, and the like, which are equipped with a web browser.

A server device may be a server that processes information by communicating with an external device and may include an application server, a computing server, a database server, a file server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet terminal (Wibro) terminal, and a wearable device such as a timepiece, a ring, a bracelet, an anklet, a necklace, glasses, a contact lens, or a head-mounted device (HMD).

A bipolar current delivery apparatus according to an embodiment of the present disclosure includes an electrode unit including a plurality of first electrodes of a needle-shaped and a plurality of second electrodes of a needle-shaped which are inserted into or in contact with a skin, an energy supply unit that applies a first current of a bipolar type to the first electrodes and applies a second current of a bipolar type to the second electrodes, and a control unit that controls the energy supply unit such that a first frequency for the first current and a second frequency for the second current are set independently of each other. This independent setting of different frequencies allows for targeted treatment of different skin layers or conditions simultaneously or sequentially.

The bipolar current delivery apparatus may deliver electric energy to the entire skin layer during a procedure, thereby enhancing the therapeutic effect. Moreover, the bipolar current delivery apparatus may provide electrical energy having various patterns by simultaneously or alternately applying currents of various frequencies to the skin, thereby bringing about various therapeutic effects.

Hereinafter, a bipolar current delivery apparatus will be described in detail.

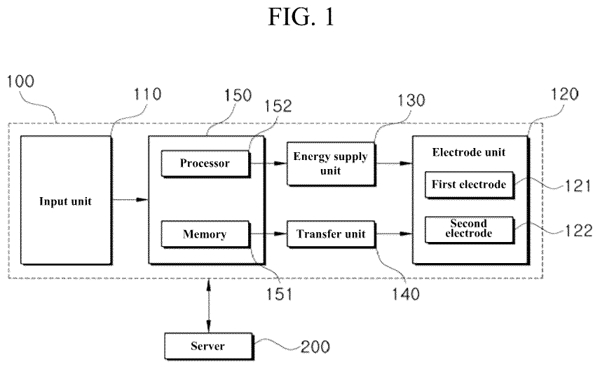

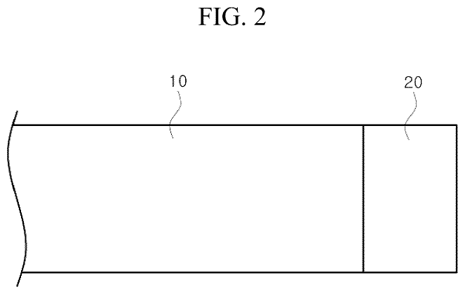

FIG. 1 is a drawing showing a configuration of a bipolar current delivery apparatus, according to an embodiment of the present disclosure. FIG. 2 is a drawing showing an electrode unit of FIG. 1 provided in cartridge housing coupled to a handpiece.

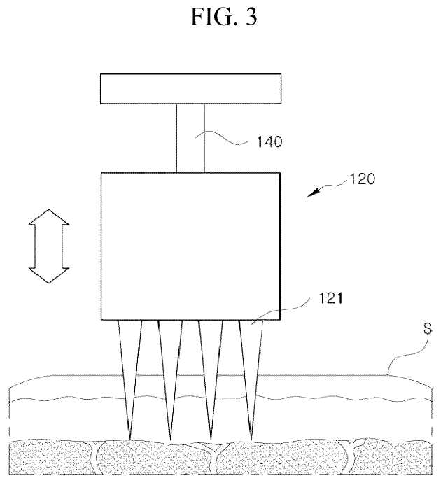

FIG. 3 is a drawing showing an example of a process in which a plurality of electrodes of a needle-shaped invades the inside of a skin by movement of the transfer unit of FIG. 1.

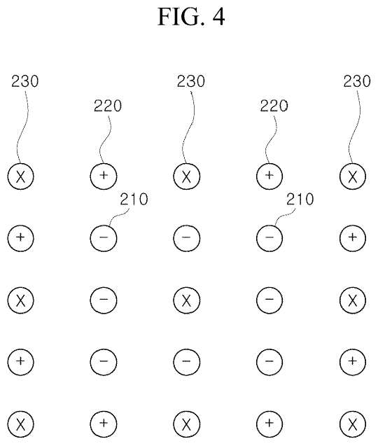

FIG. 4 is a drawing showing an embodiment of the placement of a plurality of electrodes included in the electrode unit of FIG. 1.

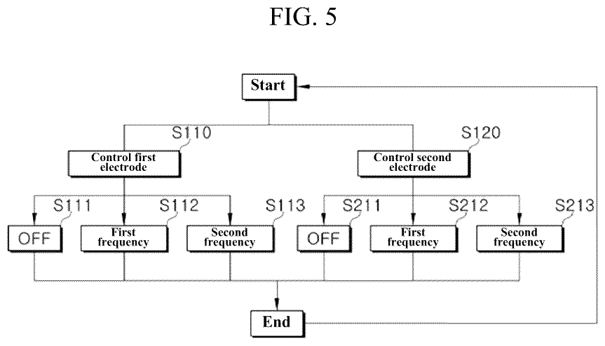

FIG. 5 is a drawing showing an example of a process of applying a first current and a second current of a bipolar type to a plurality of electrodes of the needle-shaped of FIG. 4.

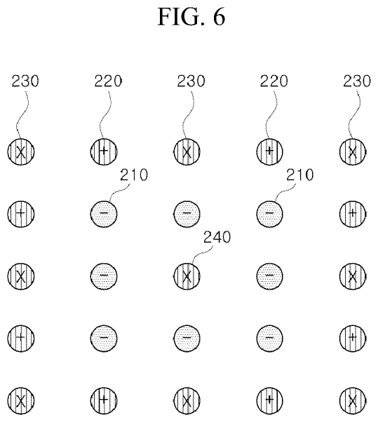

FIGS. 6 to 14 are diagrams showing various current and frequency patterns that are capable of being applied by a bipolar current delivery apparatus according to an embodiment of the present disclosure.

Referring to FIGS. 1 to 14, a bipolar current delivery apparatus 100 through skin contact may include an input unit 110, an electrode unit 120, an energy supply unit 130, a transfer unit 140, and a control unit 150.

The input unit 110 may receive procedure condition information and target depth information within a skin from a user. When the procedure condition information and the target depth information within the skin are received, the control unit 150 may control an operation of the present apparatus so as to correspond to the received procedure condition information and the received target depth information within the skin. Here, the target depth information within the skin may be a target depth value within a dermis layer, and the procedure condition information may be a procedure condition for removing wrinkles, a procedure condition for restoring skin elasticity, or a procedure condition for removing sebum.

The input unit 110 may include a hardware-type physical key (e.g., a button, a dome switch, a jog wheel, or a jog switch that is located on at least one of the front, back, and sides of the present apparatus) and a software-type touch key. For example, the touch key may consist of a virtual key, a soft key, or a visual key displayed on a touch screen-type display unit through software processing or may consist of a touch key positioned on a portion other than a touch screen. In the meantime, the virtual key or the visual key may be displayed on the touch screen while having various shapes. For example, the virtual key or visual key may be formed of graphics, texts, icons, video, or a combination thereof.

The electrode unit 120 may be provided in cartridge housing 20 coupled to a handpiece 10. The electrode unit 120 may include a plurality of electrodes 121 of needle-shaped to be inserted into a skin ‘S’. However, an embodiment is limited thereto. For example, the plurality of electrodes 121 may apply a current while being in contact with the surface of the skin ‘S’ rather than being inserted into the skin ‘S’.

The plurality of electrodes 121 of a needle-shaped may invade a deep portion (e.g., a dermal layer) of the skin ‘S’, may remove damaged collagen, elastic fibers, or the like in the deep portion of a target area by using heat generated by at least one of a low-frequency current and a high-frequency current, and may promote new formation.

The electrode unit 120 may include a plurality of different-type electrodes. In detail, the electrode unit 120 may include a first electrode 220 and a second electrode 230.

As illustrated in FIG. 4, each of the first electrode 220 and the second electrode 230 may be disposed in a form of a plurality of groups. The first electrode 220 and the second electrode 230 may each have polarity (+), but a current applied through the first electrode 220 and a current applied through the second electrode 230 may be controlled independently.

The energy supply unit 130 may apply a first current of a bipolar type to the plurality of first electrodes 220 and may apply a second current of a bipolar type to the plurality of second electrodes 230. The energy supply unit 130 may include a switching circuit for changing the frequency of the first current and the second current.

The control unit 150 controls the currents applied to the first electrode 220 and the second electrode 230. The control unit 150 may include at least one processor 152 and a memory 151. The memory 151 may store program instructions that, when executed by the processor 152, cause the processor 152 to perform the operations described herein. Here, each of the memory 151 and the processor 152 may be implemented as separate chips. Moreover, the memory 151 and the processor 152 may be implemented as a single chip.

The memory 151 may store data that supports various functions of the present apparatus, and a program for operations of the control unit, may store input/output data, and may store a plurality of application programs (or applications) running on the present apparatus, pieces of data for operations of the present apparatus, and instructions. At least part of the application programs may be downloaded from an external server through wireless communication.

The memory 151 may include the type of a storage medium of at least one of a flash memory type, hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory 151 may be separate from the present apparatus, but may be a database connected by wire or wirelessly.

The memory 151 may store depth information about a skin layer and mapping information for mapping a predetermined procedure condition set to the depth of the skin layer.

Here, the fact that the applied current is independently controlled means that the current and frequency applied through one of the first electrode or the second electrode do not affect the current and frequency applied through the other. That is, the control unit 150 sets the current applied to each of the first electrode and the second electrode.

In this specification, an electrode marked with ‘+’ means the first electrode, and an electrode marked with ‘X’ means the second electrode. That is, ‘+’ and ‘X’ marked on the electrode do not define the polarity of the electrode, and each of the first electrode and the second electrode has polarity (+).

Returning to FIG. 4, the first electrode 220 and the second electrode 230 are placed alternately with each other, and may be placed with a specific interval between the electrodes. In this way, the bipolar current delivery apparatus according to an embodiment of the present disclosure evenly distributes energy applied to the skin.

In the meantime, the bipolar current delivery apparatus according to an embodiment of the present disclosure may further include a ground electrode 210 serving as a ground part. The plurality of ground electrodes 210 may be placed in the cartridge housing 20 and may serve as a ground part while being inserted into the skin or coming into contact with the skin.

In an embodiment, the ground electrode 210 may be placed in a central portion of the cartridge housing 20, and the first electrode 220 and the second electrode 230 may be placed at edges of an area where the ground electrode 210 is positioned.

In an embodiment, the first electrode 220 and the second electrode 230 may be placed alternately with each other.

Referring to FIG. 5, the control unit 150 controls the current applied through the first electrode and the second electrode by independently performing operation S110 of controlling a current applied to the first electrode and operation S120 of controlling a current applied to the second electrode. In this specification, the current applied to the first electrode 220 is referred to as a “first current”, and the current applied to the second electrode 230 is referred to as a “second current”.

In controlling the first current applied to the first electrode, the control unit 150 may not apply a current through the first electrode (S111), may apply a current having a first frequency (S112), or may apply a current having a second frequency (S113).

In the meantime, in controlling the second current applied to the second electrode, the control unit 150 may not apply a current through the second electrode (S211), may apply a current having the first frequency (S212), or may apply a current having the second frequency (S213).

Hereinafter, the various current patterns that the control unit 150 is capable of applying through the energy supply unit 130 will be described with reference to attached drawings. Hatching shown on electrodes in FIGS. 6 to 14 indicates the frequency of the current applied to an electrode. That is, when pieces of hatching shown on a plurality of electrodes are the same as each other, it means that a current of the same frequency is applied to the corresponding electrodes. On the other hand, when there is no hatching on an electrode, it means that no current is applied. Also, for convenience, it is described that the second frequency is greater than the first frequency.

Referring to FIG. 6, the current of the first frequency may be applied to the first electrode 220 and the second electrode 230. In other words, the current of the same frequency may be applied to all electrodes except the ground electrode 210.

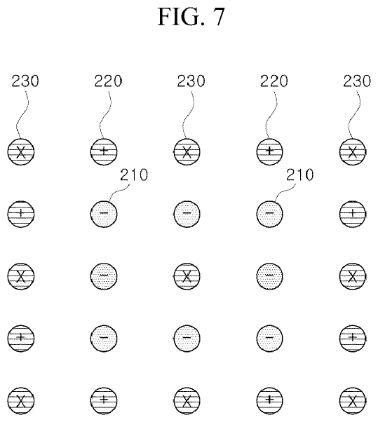

Referring to FIG. 7, the current of the second frequency may be applied to the first electrode 220 and the second electrode 230. That is, a frequency greater than the frequency shown in FIG. 6 may be applied to all electrodes except the ground electrode 210.

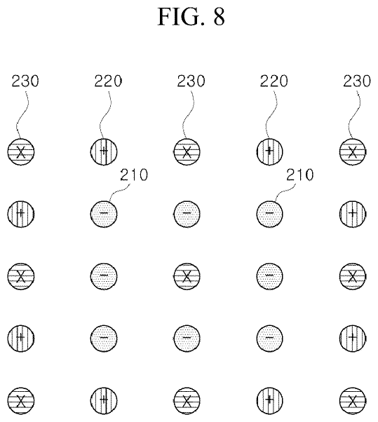

Referring to FIG. 8, a current of the first frequency may be applied to the first electrode 220, and a current of the second frequency may be applied to the second electrode 230. In other words, a low-frequency current may be applied to the first electrode 220, and a high-frequency current may be applied to the second electrode 230.

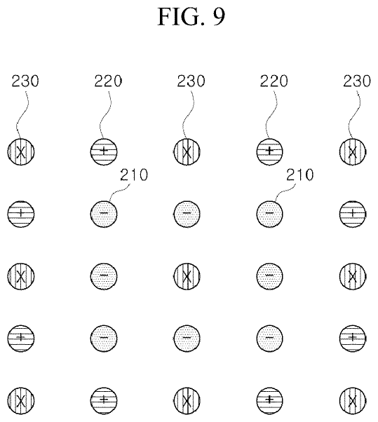

Referring to FIG. 9, a current of the second frequency may be applied to the first electrode 220, and a current of the first frequency may be applied to the second electrode 230. In other words, a high-frequency current may be applied to the first electrode 220, and a low-frequency current may be applied to the second electrode 230.

As described in FIGS. 6 to 9, the control unit 150 may control the energy supply unit 130 such that a current is simultaneously applied to the first electrode and the second electrode.

In the meantime, the control unit 150 may control the energy supply unit 130 such that a current is alternately applied to the first electrode 220 and the second electrode 230.

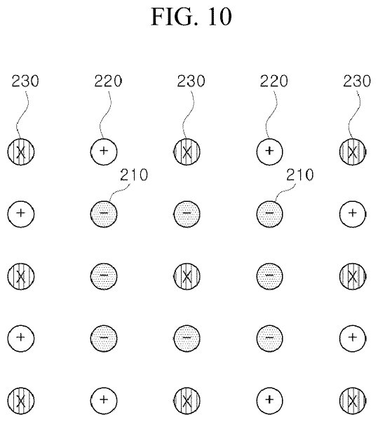

Referring to FIG. 10, no current may be applied to the first electrode 220, and a current of the first frequency may be applied to the second electrode 230.

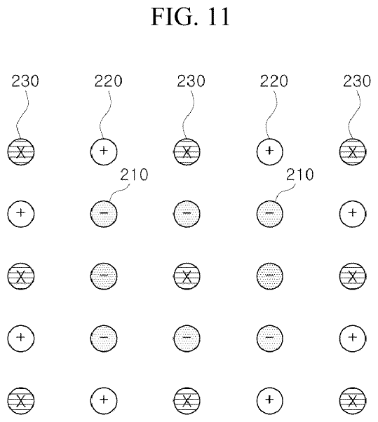

Referring to FIG. 11, no current may be applied to the first electrode 220, and a current of the second frequency may be applied to the second electrode 230.

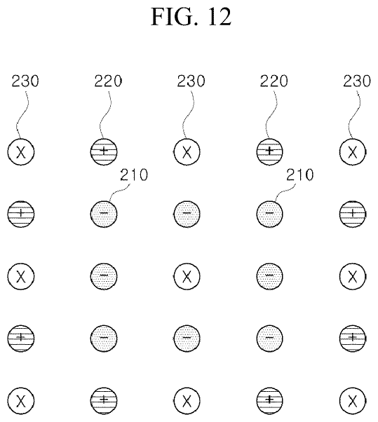

Referring to FIG. 12, the current of the first frequency may be applied to the first electrode 220, and no current may be applied to the second electrode 230.

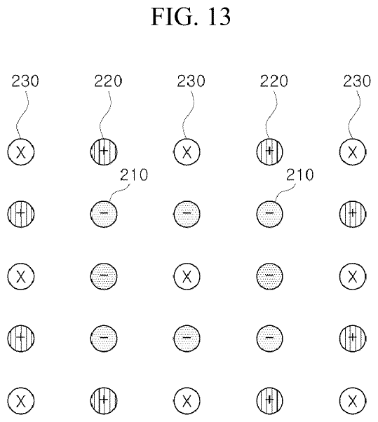

Referring to FIG. 13, the current of the second frequency may be applied to the first electrode 220, and no current may be applied to the second electrode 230.

The control unit may be switched from one of the states of FIGS. 10 and 11 to one of the states of FIGS. 12 and 13 such that a current is alternately applied to the first electrode 220 and the second electrode 230.

In the meantime, the bipolar current delivery apparatus according to an embodiment of the present disclosure may further include at least one switching electrode 240 of a needle-shaped that is inserted into or in contact with the skin.

The control unit 150 may control the energy supply unit 130 such that the current applied to the switching electrode 240 is changed from one of the first current and the second current to the other. In other words, the control unit 150 may utilize the switching electrode 240 as the first electrode or the second electrode depending on situations.

In an embodiment, the switching electrode 240 may be placed in the central portion of an area where the ground electrode 210 is placed.

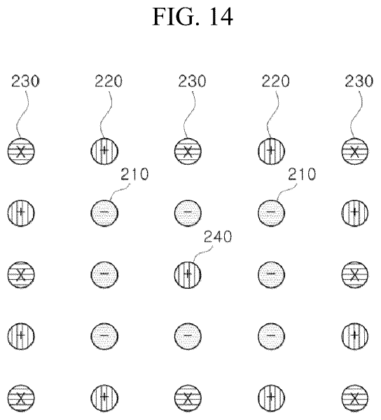

As illustrated by comparing the embodiments of FIGS. 8 and 14, the switching electrode 240, located in the central portion, can be configured to function as the second electrode (as in FIG. 8) or as the first electrode (as in FIG. 14). The bipolar current delivery apparatus according to an embodiment of the present disclosure may diversify the patterns of a current and a frequency that are capable of being applied to a skin, by including a switching electrode capable of switching an electrode type.

In an embodiment, the waveform of the current applied to each of the first electrode and the second electrode may be at least one of a sine wave, a square wave, and a pulse wave. Accordingly, currents of different waveforms may be applied to the first electrode and the second electrode, respectively.

In an embodiment, the intensity of currents applied to the first electrode and the second electrode may be different from each other.

In an embodiment, the intensity of current applied to each of the first and second electrodes may range from 0.01 mA to 1 A. The power applied to each electrode may range from 0.1 W to 50 W. The voltage level applied to each electrode may range from 1 V to 1 kV.

At least one component may be added or deleted to correspond to the performance of the components illustrated in FIGS. 1 to 4. Furthermore, it will be easily understood by those skilled in the art that mutual locations of the components may be changed to correspond to the performance or structure of the system.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

According to the above-mentioned problem solving means of the present disclosure, an apparatus may deliver electric energy to the entire skin layer during a procedure, thereby enhancing the therapeutic effect.

Moreover, a bipolar current delivery apparatus according to an embodiment of the present disclosure may provide electrical energy having various patterns by simultaneously or alternately applying currents of various frequencies to the skin, thereby bringing about various therapeutic effects.

Effects of the present disclosure are not limited to the effects mentioned above, and other effects not mentioned will be clearly understood by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A bipolar current delivery apparatus comprising:
an electrode unit including a plurality of first electrodes of a needle-shaped and a plurality of second electrodes of a needle-shaped which are inserted into or in contact with a skin;
an energy supply unit configured to apply a first current of a bipolar type to the first electrodes and to apply a second current of a bipolar type to the second electrodes; and
a control unit configured to control the energy supply unit such that a first frequency for the first current and a second frequency for the second current are set independently of each other,
wherein the first frequency and the second frequency are different from each other.

2. The bipolar current delivery apparatus of claim 1, wherein the control unit is configured to:
control the energy supply unit such that a current is simultaneously applied to the first electrodes and the second electrodes.

3. The bipolar current delivery apparatus of claim 1, wherein the control unit is configured to:
control the energy supply unit such that a current is alternately applied to the first electrodes and the second electrodes.

4. The bipolar current delivery apparatus of claim 3, further comprising:
a plurality of ground electrodes of a needle-shaped that are inserted into or in contact with a skin,
wherein the first electrodes and the second electrodes are placed at edges of an area where the ground electrodes are placed.

5. The bipolar current delivery apparatus of claim 4, wherein the first electrodes and the second electrodes are placed alternately with each other.

6. The bipolar current delivery apparatus of claim 5, further comprising:
at least one switching electrode of a needle-shaped that is inserted into or in contact with a skin,
wherein the control unit is configured to:
control the energy supply unit such that a current applied to the switching electrode is changed from one of the first current and the second current to the other.

7. The bipolar current delivery apparatus of claim 6, wherein the switching electrode is placed in a central portion of the area where the ground electrodes are placed.

8. The bipolar current delivery apparatus of claim 7, wherein a waveform of a current applied to the first electrodes and the second electrodes is at least one of a sine wave, a square wave, and a pulse wave.

9. The bipolar current delivery apparatus of claim 8, wherein the intensity of the first current is different from the intensity of the second current.

10. A method performed by a bipolar current delivery apparatus including a plurality of first electrodes of a needle-shaped and a plurality of second electrodes of a needle-shaped which are inserted into or in contact with a skin, the method comprising:
setting a first frequency for a first current applied to the first electrodes;
applying a first current of a bipolar type to the first electrodes;
setting a second frequency for a second current applied to the second electrodes; and
applying a second current of a bipolar type to the second electrodes,
wherein the first frequency and the second frequency are independently set to different frequencies.
